(19) United States
(12) Patent Application Publication
(10) Pub. No.: US 2010/0312068 A1
(43) Pub. Date: Dec. 9, 2010

(54) Surgical retractor

(76) Inventor: Brian E. Dalton, Erie, PA (US)

Correspondence Address: FLOYD B. CAROTHERS;CAROTHERS AND CAROTHERS, 445 FORT PITT BOULEVARD, SUITE 200, PITTSBURGH, PA, 15219, US

(21) Appl. No.: 12/455,794

(22) Filed: Jun. 8, 2009

(51) Int. Cl. A61B 1/32

(52) U.S. Cl. 600/210

(57) ABSTRACT

The surgical retractor of the present invention includes a proximal base frame having an opening for overlying an operative site on a patient. A plurality of tissue engaging retractor blades are arrayed and disposed about and extend downwardly from the frame about a central axis of the opening to distal blade ends. Each blade has an outwardly and generally horizontally extending blade handle at their proximal ends which handles are slidably mounted to the frame whereby the blades may be selectively retracted by the handles from the central axis for thereby retracting tissue. The blade handles pass through guide slots in the frame, and more particularly through guide slots provided in respective pivotal segments that are pivotally secured to the frame on respective horizontal axes thereby the blades may be thereby independently pivoted downwardly and outwardly from the central axis. An internally opened expansion collet is coaxially received in the top of the frame for adjustable coaxial advancement downwardly into the frame. The collet is dimensioned for simultaneous engagement of selected of the pivotal segments for thereby simultaneously urging them together with their respective blades downwardly and outwardly away from the central axis to further retract tissue.

### BACKGROUND OF THE INVENTION

The present invention relates to expandable retractors for use in surgery. More particularly, the present inventions relates to mechanical selectively expandable retractors for removing subsurface tissue in surgery performed percutaneously through a stab incision for performing minimally invasive surgical techniques.

Traditional open surgical procedures performed on locations deep within the body can cause significant trauma to the intervening tissues. Such procedures often require a long incision, extensive muscle stripping, prolong retraction of tissues, denervation and devascularication of tissue. These traditional open surgeries can require extensive operating time and extensive post-operative recovery time, and in some cases, can lead to permanent scarring and more severe pain to the patient.

Accordingly, minimally invasive surgical procedures are preferred. Such minimal access procedures require a device to expose the operable field with a minimal of trauma to the surrounding tissues, most notably musculature. These minimal access devices or retractors generally rely on splitting muscle planes to reach desired operative fields. Accordingly, this minimally invasive approach is more desirable than standard surgical operations which rely on stripping muscle of its attachments. It is an object of the present invention to provide a retractor for minimally invasive surgery which eliminates the requirement of multiple steps to dilate the musculature, or most notably eliminates the requirement or use of a “tube over tube” sequential dilation system, as well as the elimination of the adaptation and use of standard retractor systems for minimal access surgery.

Many surgical retractors employ a circular array of downwardly depending retractor blades, each of which has a handle that is slidably secured to an open base ring or frame whereby the retractor handles may be selectively retracted horizontally outward relative to the base ring for retraction of tissue. For example, see U.S. Pat. Nos. 7,344,495 and 7,435,219, and US Patent Application Publication Nos. 2007/0238932; 2007/0156026; 2007/0156025 and 2009/0018401. In all of these surgical retractors, the retractor blades may be independently horizontally moved outward, and the blades may also be independently pivoted downward and outward. However, a deficiency incurred is that when the blades are pivoted or tilted, they must be done so independently which is not easy or always functional under operating conditions. In addition, the selective removal or addition of surgical blades to and from the assembly is not readily or easily accomplished.

### SUMMARY OF THE INVENTION

The surgical retractor of the present invention includes a proximal base frame having an opening for overlying an operative site on a patient. A plurality of tissue engaging retractor blades are disposed in an array about a central axis of the opening. The retractor blades extend downwardly about the axis to distal blade ends. Each blade is provided with an outwardly and generally horizontally extending blade handle at the proximal ends of the blades, and these handles are slidably mounted to the frame whereby the blades may selectively retracted by the handles from the central axis for thereby retracting tissue.

The blade handles pass through guide slots in the frame. The guide slots are respectively provided in pivotal segments that are pivotally secured to the frame on respective horizontal axes whereby the blades may be independently pivoted outward from the central axis. An internally open expansion collet is coaxially received in the top of this frame for adjustable coaxial advancement downwardly into the frame. The collet is dimensioned for simultaneously engaging preselected of these pivotal segments, thereby urging them to pivot simultaneously whereby the selected blades will thereby simultaneously pivot or tilt downwardly and outwardly away from the central axis to retract tissue. Advantages are that retractor blades may be easily removed or added as desired, and in addition, the retractor of the present invention eliminates the requirement of multiple steps of independently tilting each retractor blade to dilate the musculature.

Ratchet engagement is provided between the pivotal segments and the blade handles for adjustably securing the horizontal advancement and retraction of the blades.

Each of the pivotal segments is provided with an inwardly projecting contact protrusion for engaging the bottom advancing end of the collet for pivoting the segments on their axis. Respective protrusions may be removed for blades that are not to be pivoted. The collet is threadably engaged with the base frame to thereby threadably advance the collet downward by turning it clockwise relative to the frame.

The horizontal extending retractor blade handles engage the downwardly extending retractor blades through an angular handle section. These beveled portions of the handles allow greater visualization when the retractor blades are pivoted down and thereby outwardly, as otherwise the retractor blade handles, where they connect to the upper or posterior portions of the retractor blades, tend to protrude medially and obscure the view unless this bevel is in place.

### BRIEF DESCRIPTION OF THE DRAWINGS

Other objects and advantages appear hereinafter in the following description and claims. The accompanying drawings show, for the purpose of exemplification, without limiting the scope of the invention or appended claims, certain practical embodiments of the present invention wherein:

FIG. 1 is an isometric view of the proximal base frame portion of the surgical retractor of the present invention;

FIG. 2 is a top view of the base frame illustrated in FIG. 1;

FIG. 3 is a view in front elevation of the expansion collet portion of the surgical retractor of the present invention;

FIG. 4 is a view in front elevation of the assembled surgical retractor of the present invention without the inclusion of the retractor blades and with the base frame shown in vertical cross section as seen along section line IV-IV;

FIG. 5 is a view of the surgical retractor of the present invention as shown in FIG. 4 with the inclusion of the left and right side retractor blades only;

FIG. 6 is a view of the surgical retractor of the present invention identical to that illustrated in FIG. 5, illustrating the retractor blades in a pivoted or tilted position for additionally retracting tissue;

FIG. 7 is a top view of an array of four retractor blades for utilization in the surgical retractor of the present invention illustrated in FIGS. 4, 5 and 6;

FIG. 8 is a view in front elevation of the retractor blade array shown in FIG. 7;

FIG. 9 is a view in side elevation of one of the four blade pivotal segments shown in FIGS. 4, 5 and 6;

FIG. 10 is a front view of the pivotal segment shown in FIG. 9;

FIG. 11 is a rear view of the pivotal segment shown in FIG. 9 with the spring loaded keeper removed for clarity;

FIG. 12 is a view in side elevation of the pivotal segment shown in FIG. 9 with the retractor blade handle removed and with a pivotal contact protrusion added;

FIG. 13 is a rear view of the contact protrusion of FIG. 12;

FIG. 14 is a view in side elevation of the contact protrusion shown in FIG. 13;

FIG. 15 is a front view of the contact protrusion shown in FIGS. 13 and 14;

FIG. 16 is a top view of an alternative array of retractor blades utilized in the surgical retractor of the present invention;

FIG. 17 is a view in side elevation of the left hand retractor blade shown in FIG. 16; and

FIG. 18 is a view in side elevation of the upper most rearward retractor blade shown in FIG. 16.

### DETAILED DESCRIPTION OF THE PREFERRED EMBODIMENTS

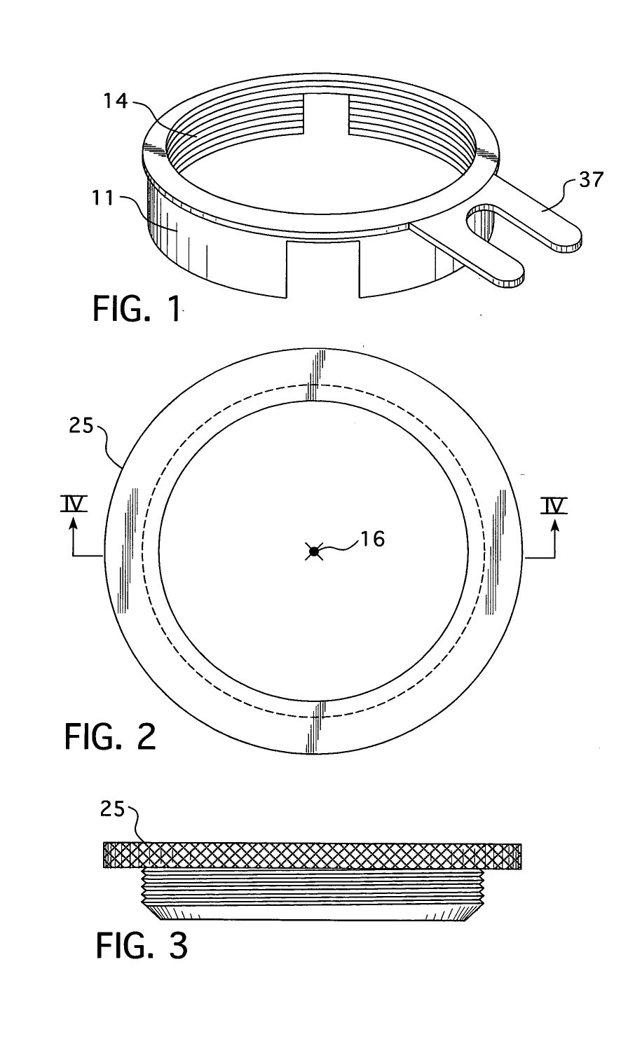

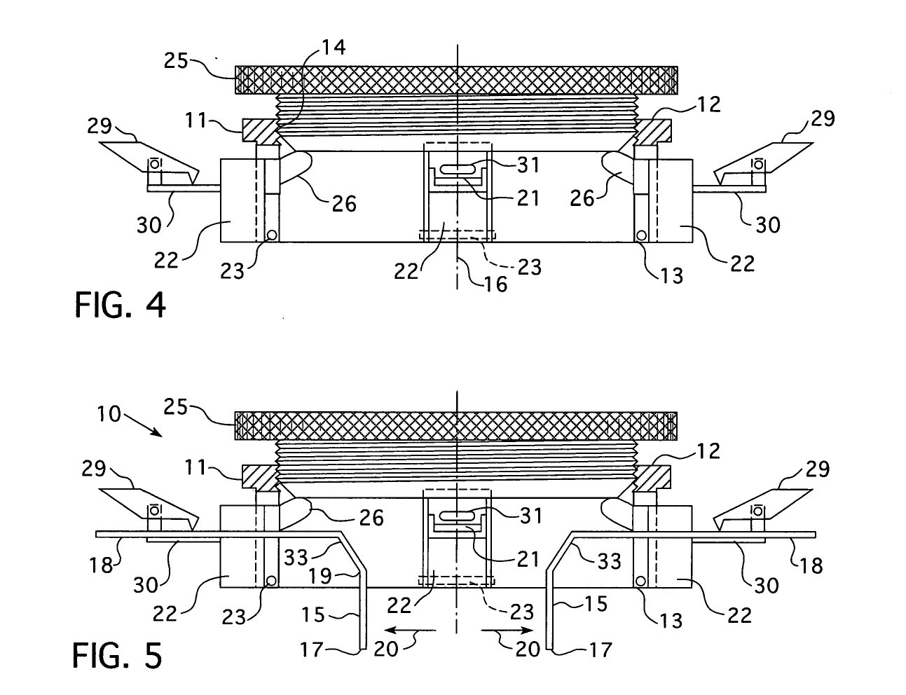

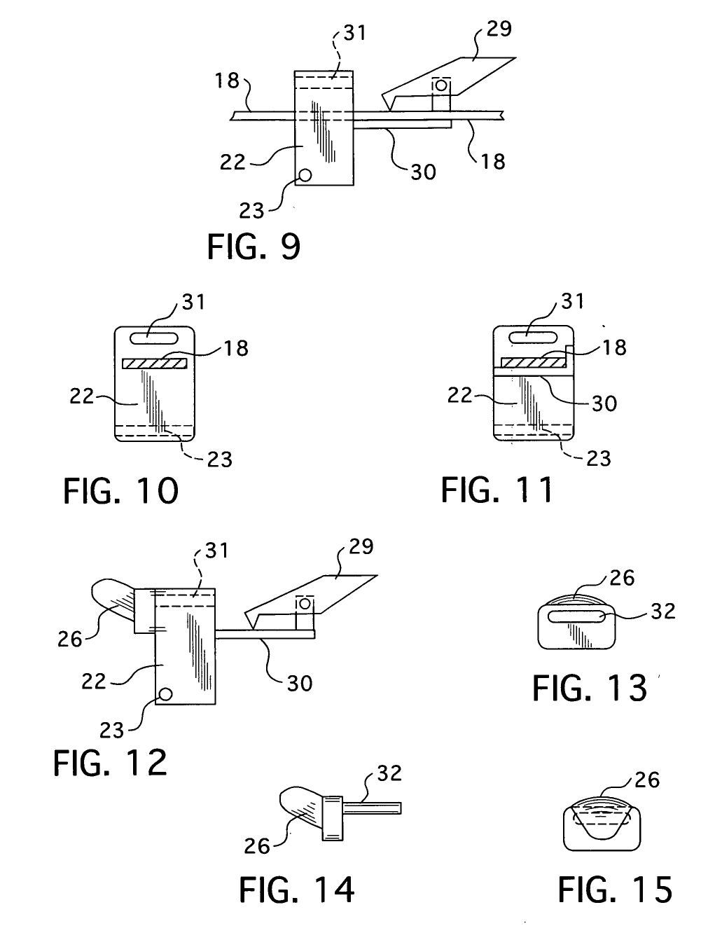

Referring first to the first embodiment of the present invention, reference is made to FIGS. 1 through 15.

The surgical retractor 10 of the present invention is comprised of a proximal base frame 11 having a top 12 and a bottom 13 and an opening 14 for overlying an operative site on a patient. The retractor 10 is provided with a plurality of tissue engaging retractor blades 15 disposed in an array about central axis 16. The retractor blades 15 also extend downwardly from frame 11 about central axis 16 to their distal ends 17. Each blade 15 is provided with an outwardly and generally horizontally extending blade handle 18 at the proximal ends 19 of the blades. The handles 18 are slidably mounted to frame 11 whereby the blades may be selectively retracted by the handles 18 from central axis 16 as illustrated by the arrows 20 for retracting tissue.

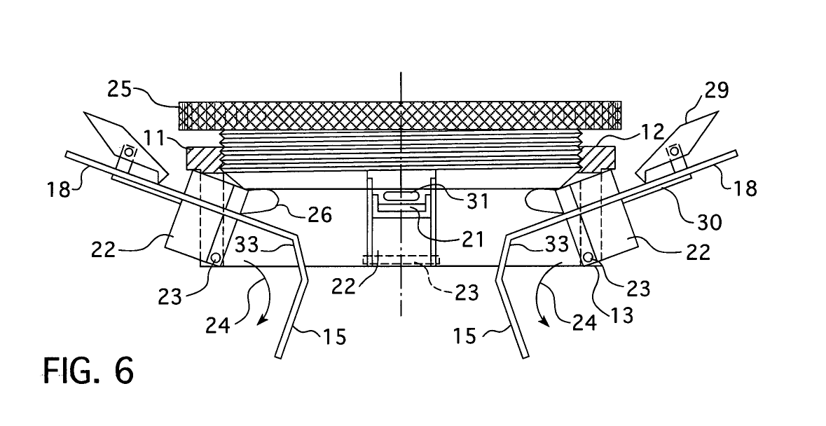

Blade handles 18 pass through respective guide slots 21 of frame 11. Actually the guide slots 21 are provided in respective pivotal segments 22 which are respectively secured to frame 11 on respective horizontal axes 23. Four such pivotal segments 22 are illustrated and arranged equidistance around frame 11. However, more or less of such segments may be provided. The blades 15 may thus be independently pivoted outward from axis 16 as illustrated by arrows 24 in FIG. 6.

Internally open expansion collet 25 is coaxially and threadably received at the top 12 of frame 11 in opening 14 for adjustable coaxial advancement downwardly into frame 11. Collet 25 is further beveled on the bottom thereof and dimensioned for simultaneously engagement of the removable contact protrusions 26 of pivotal segments 22 for thereby urging the segments 22 to pivot as indicated by arrows 24 in FIG. 6, together with respective of their blades 15, downwardly and outwardly away from the central axis 16 to retract tissue.

Each of the blade handles 18 are provided with a series of ratchet teeth 28 on their upper surfaces for adjustably securing the horizontal advancement and retraction of blades 15. The ratchet teeth are engaged by spring loaded keepers 29 which are hingedly secured to the respective pivotal segments 22 on supports 30 and contact respective ratchet teeth 28 on the handles under spring pressure.

Each of the pivotal segments 22 is provided with a slot 31 for receiving the stem 32 of one of the contact protrusions 26. Thus the contact protrusions 26 may be selectively provided or not provided on each of the pivotal segments 22. Accordingly, if it is not desired to have that particular pivotal segment 22 to rotate about its respective axis 23 when the collet 25 is advanced downwardly into frame 11, then that particular pivotal segment is not provided with a contact protrusion 26.

Each of the handles 18 for respective retractor blades 15 is provided with a beveled or angular handle section 33 whereby when the respective blades 15 are pivoted about the respective pivotal axes 23 for pivotal sections 22, as indicated by arrows 24 in FIG. 6, the angle bends between handles 18 and blades 15 do not interfere with the central view down through the opening in collet 25 as tissue is being retracted.

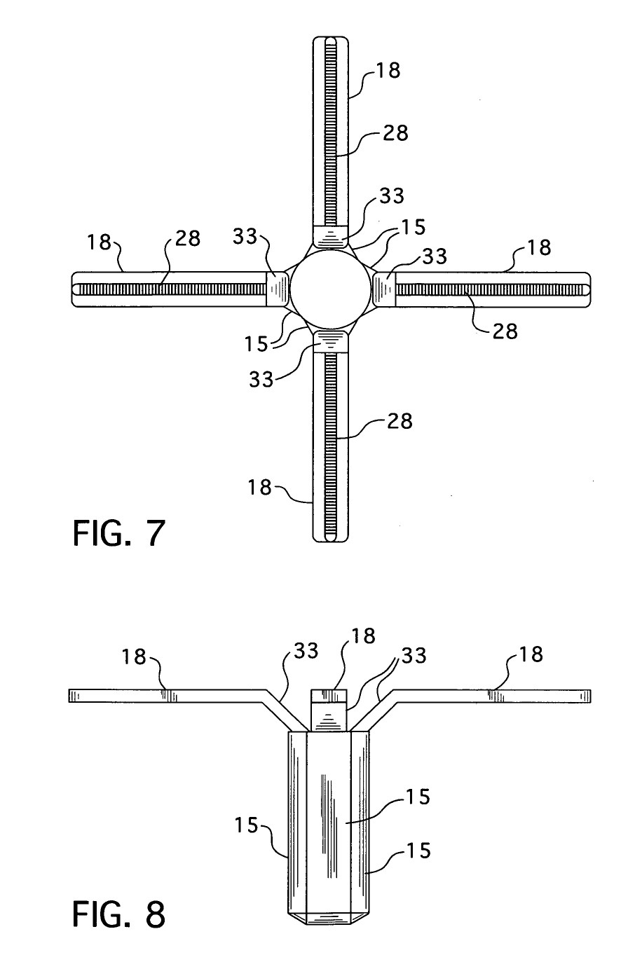

In order to illustrate typical dimensions of the retractor 10, the inside diameter of the clustered array of blades 15 shown in FIG. 7 might be selected as 22 millimeters and each of the handles 18 might be selected typically as being approximately two inches long. A typical internal diameter for the collet 25 would be four inches.

The entire retractor 10 would typically be manufactured of stainless steel for sterility purposes.

As is illustrated in FIG. 1, a retractor attachment 37 is secured to the base frame 11 so that the retractor 10 may be attached to an operating room table (not shown) by way of a flexible arm so that the retractor can be positioned and held in place during the operation procedure.

The retractor 10 of the present invention is very adaptable to different aspects of spinal surgery. For example, the medial most retractor blade up against the spinous process generally does not need to be tilted inward since it would only contact bone and this would limit the amount of tilt placed on the other retractor blades. Accordingly, the respective contact protrusion 26 for that retractor blade and its associated pivotal segment 22 may be removed or omitted. For example, the contact protrusions 26 on the pivotal segments 22 in the north and south positions of patient's body would be included thereby allowing retractions in this direction and leaving the medial and possibly the lateral retractor blades to be only horizontally retracted. Independent pivoting and securement thereafter of each blade 15 to be pivoted is not required as the blades selected for pivoting are simultaneously pivoted by the single engagement and rotation of the collet 25 down into the frame 11.

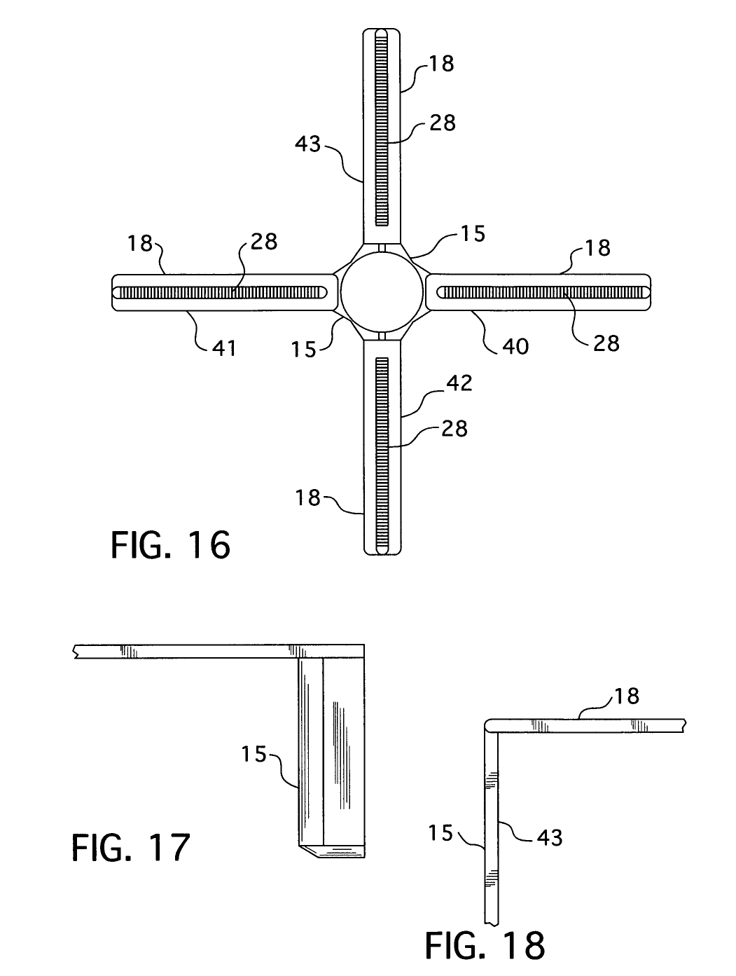

Referring next to FIGS. 16 through 18, a different embodiment of the blades 15 is illustrated. In this array, the retractor of blades 15 are not provided with the beveled section 33 for handles 18 and the right and left blades 40 and 41 are fully nested with each other while the two side blades 42 and 43 are much smaller and do not interrupt with the nesting of blades 40 and 41. When the retractor blades 40 and 41 are deployed or retracted, they increase the diameter of the central circle in ovoid fashion. The nested retractor blades are provided basically to hold back soft tissue so that it does not herniate into the expanding oval. Generally, the larger nesting retractor blades 40 and 41 would be permitted to tilt, but the flat retractor side blades 42 and 43 would not be permitted to pivot or tilt and therefore their respective pivotal segments 22 would not be provided with contact protrusions 26. This provides a desirable conical ovoid shape increasing from the skin level down to the depth of the surgical site to allow maximal exposure of a fusion within the spine or any point of interest.

I claim:

1. A surgical retractor comprising:
a proximal base frame having a top and a bottom and an opening for overlying an operative site on a patient;
a plurality of tissue engaging retractor blades disposed about and extending downwardly from said frame about a central axis of said opening to distal blade ends, and each blade having an outwardly and generally horizontally extending blade handle at their proximal end which is slidably mounted to said frame whereby said blades may be selectively retracted by said handles from said central axis for thereby retracting tissue;
said blade handles passing through guide slots in said frame, said guide slots provided in respective pivotal segments pivotally secured to said frame on respective horizontal axes whereby said blades may be thereby independently pivoted downwardly and outwardly from said central axis; and
an internally open expansion collet coaxially received in the top of said frame for adjustable coaxial advancement downwardly into said frame and dimensioned for simultaneously engaging selective ones of said pivotal segments and thereby urging them to pivot together with respective of said blades downwardly and outwardly away from said central axis to retract tissue.

2. The surgical retractor of claim 1, including ratchet engagement means between said pivotal segments and said blade handles for adjustably securing the horizontal advancement and retraction of said blades.

3. The surgical retractor of claim 1, said segments having inwardly protruding and removable contact protrusions for engaging a bottom advancing end of said collet for pivoting said segments on their axes.

4. The surgical retractor of claim 1, wherein said collet is threadably engaged with said base frame.

5. The surgical retractor of claim 1, wherein said horizontally extending retractor blade handles engage said downwardly extending retractor blades through a downwardly extending angular handle section.
